(19) United States
(12) Patent Application Publication
(10) Pub. No.: US 2012/0221034 A1
(43) Pub. Date: Aug. 30, 2012

(54) APPARATUS AND METHODS FOR REMOVING TISSUE

(75) Inventors: Fred B. Dinger, III, San Antonio, TX (US); Gabriele G. Niederauer, San Antonio, TX (US); Jeffrey S. Wrana, San Antonio, TX (US)

(73) Assignee: ENTRIGUE SURGICAL, INC., San Antonio, TX (US)

(21) Appl. No.: 13/391,587

(22) PCT Filed: Aug. 24, 2010
(86) PCT No.: PCT/US10/46462

§ 371 Date: May 16, 2012

Related U.S. Application Data
(60) Provisional application No. 61/236,774, filed on Aug. 25, 2009.

(51) Int. Cl. A61B 17/3205

(52) U.S. Cl. 606/170

(57) ABSTRACT

Apparatus and methods for removing tissue a body cavity. Apparatus and methods comprise an inner sleeve, a middle sleeve, an outer sleeve, and an irrigation conduit.

### CROSS-REFERENCE TO RELATED APPLICATION

This application claims priority to U.S. Provisional Patent Application Ser. No. 61/236,774 filed Aug. 25, 2009 and incorporated by reference herein.

### FIELD OF THE INVENTION

The present disclosure relates generally to apparatus, systems and methods for shaving, cutting, or otherwise removing tissue or other material, particularly from a cavity in the ear, nose, and throat (ENT) regions. The present disclosure relates more specifically to apparatus, systems and methods that comprise a device that provides irrigation and includes an articulating and/or extending blade for shaving, cutting, or otherwise removing tissue or other material from a cavity in ENT regions.

### BACKGROUND INFORMATION

In certain instances, it may be desirable to remove tissue or other material from an ear, nose, or throat (ENT) cavity. In certain circumstances, the tissue or other material may be difficult to access. Therefore, it may be desirable to have an apparatus that can articulate in order to reach difficult to access material. It may also be desirable to have an apparatus that can extend or retract a blade from an articulatable shaft that is configured for insertion into an ENT cavity.

### SUMMARY

Exemplary embodiments of the present disclosure comprise apparatus and methods for removing material from a body cavity (e.g., an ear, nose, or throat [ENT] cavity). In certain embodiments, the apparatus may comprise an inner sleeve, a middle sleeve, an outer sleeve, and an irrigation conduit. The outer sleeve may comprise a continuous outer wall that provides a sealed environment for irrigation fluid.

Certain embodiments comprise an apparatus for removing tissue from a body cavity, where the apparatus may comprise: an inner sleeve; a middle sleeve comprising an aperture that exposes a portion of the inner sleeve; an outer sleeve; and an irrigation conduit. In specific embodiments, the irrigation conduit may comprise a first end proximal to the aperture of the middle sleeve, and the inner sleeve may be configured to move relative to the middle sleeve. In certain embodiments, the irrigation conduit may be disposed between the middle sleeve and the outer sleeve. In particular embodiments, the irrigation conduit may be integral to the outer sleeve. In specific embodiments, the outer sleeve may comprise a flexible portion proximal to the aperture of the middle sleeve. In certain embodiments, the outer sleeve may comprise a continuous wall in the flexible portion.

In particular embodiments, the outer sleeve may comprise polyethylene, polypropylene, or PTFE. In specific embodiments, the inner sleeve may rotate within the middle sleeve. In certain embodiments, the inner sleeve may move axially within the middle sleeve. In particular embodiments, the inner sleeve may comprise a blade exposed by the aperture. In certain embodiments, the aperture may comprise a sharp edge. In specific embodiments, the outer sleeve may comprise a control member configured to articulate the outer sleeve, the middle sleeve and the inner sleeve. In certain embodiments, the control member may include a cable, wire or ribbon.

Particular embodiments may comprise a method of removing tissue from a body cavity, where the method includes providing an apparatus comprising: an inner sleeve; a middle sleeve comprising an aperture that exposes a portion of the inner sleeve; an outer sleeve; and an irrigation conduit. The irrigation conduit may comprise a first end proximal to the aperture of the middle sleeve, and the inner sleeve may be configured to move relative to the middle sleeve. Specific embodiments of the method may also comprise: inserting the apparatus into the body cavity; excising tissue by engaging tissue between the inner sleeve and the middle sleeve; irrigating the body cavity with fluid from the irrigation conduit; and removing the excised tissue. In particular embodiments, the method may further comprise providing suction to the inner sleeve. In certain embodiments, the body cavity may be an ear, nose or throat cavity.

Certain embodiments may comprise rotating the inner sleeve relative to the middle sleeve and/or moving the inner sleeve axially relative to the middle sleeve. In particular embodiments, the method may comprise articulating the outer sleeve, the middle sleeve and the inner sleeve. In specific embodiments, the outer sleeve may comprise a control member, and the outer sleeve, the middle sleeve and the inner sleeve may be articulated via the control member, which may comprise a cable, wire or ribbon.

### BRIEF DESCRIPTION OF THE DRAWINGS

FIG. 1 illustrates a perspective view of an apparatus according to a first embodiment of the present disclosure.

FIG. 2 illustrates a detailed perspective view of the apparatus of FIG. 1.

FIG. 3 illustrates a detailed perspective view of the apparatus of FIG. 1.

FIG. 4 illustrates a detailed perspective view of the apparatus of FIG. 1 in a flexed position.

### DETAILED DESCRIPTION OF EXEMPLARY EMBODIMENTS

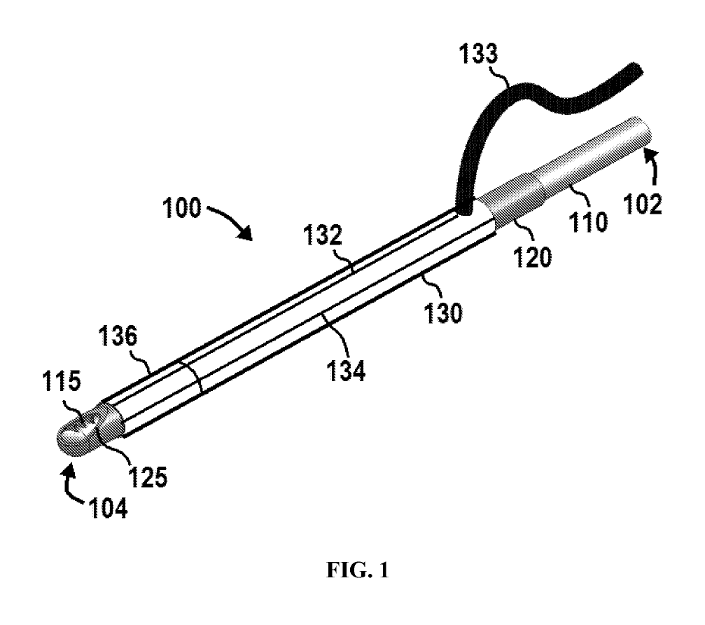

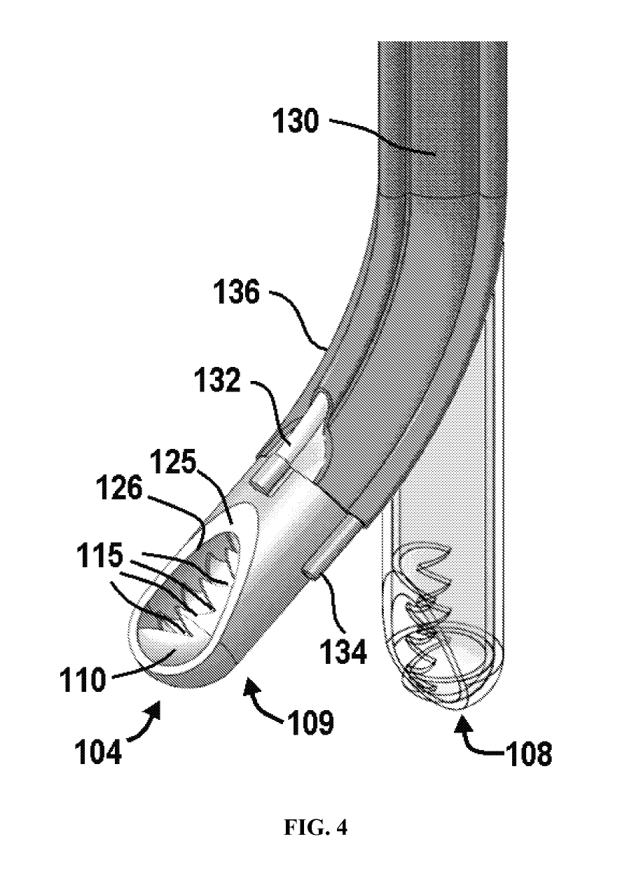

Referring now to the exemplary embodiment shown in FIGS. 1-4, an apparatus 100 for removing tissue comprises an inner sleeve 110, a middle sleeve 120, and an outer sleeve 130. As used herein, the term “sleeve” includes any tubular structure with an internal conduit or passage. In specific embodiments, a sleeve may be a generally cylindrical tubular structure. In the embodiment shown, apparatus 100 comprises a proximal end 102 and a distal end 104, and middle sleeve 120 comprises an aperture 125 proximal to distal end 104. In this embodiment, inner sleeve 110 comprises one or more blades 115. As used herein, the term “blade” comprises any sharp surface suitable for cutting tissue. In the exemplary embodiment shown, outer sleeve 130 comprises an irrigation conduit 132 and a control member 134. In the perspective view shown in FIG. 1, outer sleeve 130 comprises a flexible portion 136 configured to flex or articulate (as shown in FIG. 4). Control member 134 can be manipulated by a control device (not shown) to flex or move flexible portion 136, as understood by those skilled in the art. In particular exemplary embodiments, control member 134 may comprise a cable, a wire or a ribbon.

During operation, inner sleeve 110 can move relative to middle sleeve 120 so that blades 115 move relative to aperture 125. In specific exemplary embodiments, inner sleeve 110 rotates within middle sleeve 120. In other exemplary embodiments, inner sleeve 110 may move axially relative to middle sleeve 120 (e.g., along the longitudinal axis of the sleeve). In other exemplary embodiments, inner sleeve 110 may oscillate within middle sleeve 120 (e.g., move rotationally and axially). Tissue that is desired to be removed can be inserted through aperture 125 and engaged by blades 115. As inner sleeve 110 moves, blades 115 can shear or cut tissue that is engaged with blades 115 and aperture 125. In the specific embodiment shown, aperture 125 comprises a perimeter that includes a sharp edge 126 to assist in shearing or cutting the tissue.

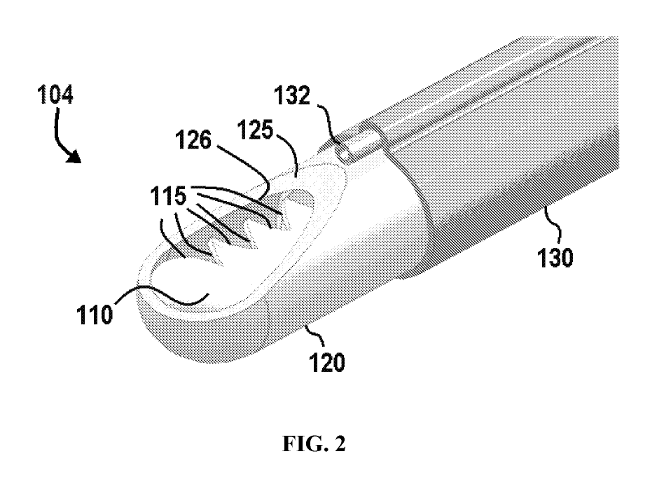

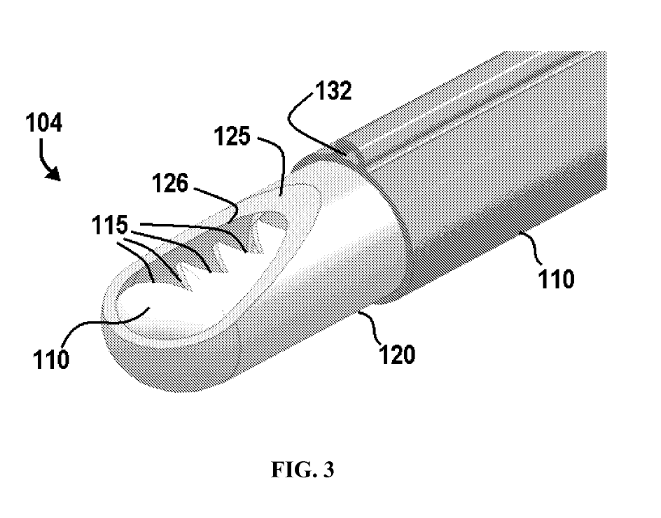

The tissue that has been cut or removed can then be drawn toward proximal end 102 via suction applied through inner sleeve 110. In addition, irrigation fluid (e.g., saline or water) can be delivered via irrigation conduit 132 to the area near aperture 125 (e.g. into the body cavity) to assist in removal of the excised tissue. The irrigation conduit 132 may be a single tube for delivering fluids or may have a plurality of tubes, e.g., one tube on either side of the sleeve in order to optimize or direct the delivery of the fluid. In the embodiment shown, irrigation conduit 132 is in fluid communication with an irrigation fluid supply line 133 which may also be in fluid communication with an irrigation fluid reservoir (not shown). As shown in FIG. 2, irrigation conduit 132 may be a separate distinct component (e.g., small diameter tubing) inserted into a channel of outer sleeve 130 (or between outer sleeve 130 and middle sleeve 120). As shown in FIG. 3, irrigation conduit 132 may be an integral conduit or channel formed in outer sleeve 130. For purposes of clarity, control member 134 is not shown in FIGS. 2 and 3.

Referring now to FIG. 4, apparatus 100 is shown (in phantom) in a first position 108 in which flexible portion 136 is straight. In addition, FIG. 4 shows apparatus 100 in a second position 109 in which flexible portion is articulated (e.g., flexed or curved). In certain embodiments, inner sleeve 110 and middle sleeve 120 comprise a series of articulated linkages (not visible in the figures due to outer sleeve 130) that allow inner sleeve 110 and middle sleeve 120 to flex. Outer sleeve 130 may be comprised of any suitable material and suitable dimensions that allows flexible portion 136 to flex. In certain embodiments, outer sleeve 130 may comprise polyethylene, polypropylene, polytetrafluoroethylene (PTFE), acrylonitrile butadiene styrene (ABS) or any suitable thermoplastic. In specific embodiments, outer sleeve 130 may be between 0.005 inches and 0.020 inches thick. In particular embodiments, outer sleeve 130 may be between 0.010 inches and 0.015 inches thick.

During operation of apparatus 100, control member 134 can be manipulated to place distal end 104 in a desired location or orientation. Blades 115 and sharp edge 126 of aperture 125 can engage and remove tissue as inner sleeve 110 moves relative to middle sleeve 120. Irrigation conduit 132 can irrigate the area by delivering fluid to the tissue cutting site. In the embodiment shown, irrigation conduit 132 comprises a flexible conduit with a continuous outer wall from fluid supply line 133 to the end of irrigation conduit 132 that is proximal to aperture 125 (e.g., the outer wall provides a sealed environment that contains fluid flowing through irrigation conduit 132). In exemplary embodiments, irrigation conduit 132 does not comprise hinged portions or linkages that may allow fluid to escape from irrigation conduit 132 prior to the fluid reaching the end proximal to aperture 124.

In addition, outer sleeve 130 comprises a flexible conduit with a continuous outer wall in the region from fluid supply line 133 to the end of irrigation conduit 132 that is proximal to aperture 125 (e.g., outer sleeve 130 provides a sealed environment in the region between aperture 125 and fluid supply line 133). In exemplary embodiments, outer sleeve 130 does not comprise hinged portions or linkages that may allow air to be drawn through outer sleeve 130. Such a configuration may be particularly useful when suction is applied to inner sleeve 110 (e.g., in an effort to remove excised tissue particles). By providing a continuous wall and a sealed environment, outer sleeve 130 can allow a vacuum or suction to be maintained and therefore allow the excised particles to be removed. Outer sleeve 130 may also provide a smooth outer surface that allows apparatus 100 to be more easily inserted and removed into a body cavity.

### EQUIVALENTS AND SCOPE

While an exemplary embodiment is described herein, it will be understood that various modifications to the method and apparatus can be made without departing from the scope of the present invention. For example, the positions of the control mechanisms may be varied from those illustrated. Furthermore, the sequential recitation of steps in any claim is not a requirement that the steps be performed in any particular order, unless otherwise so stated.

The foregoing has been a description of certain non-limiting preferred embodiments of the invention. Those skilled in the art will recognize, or be able to ascertain using no more than routine experimentation, many equivalents to the specific embodiments of the invention described herein. Those of ordinary skill in the art will appreciate that various changes and modifications to this description may be made without departing from the spirit or scope of the present invention, as defined in the following claims. For example, the positions of the control mechanisms may be varied from those illustrated.

In the claims articles such as “a”, “an”, and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that embodiments of the invention encompasses all variations, combinations, and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, where the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise. In addition, embodiments of the invention encompasses compositions made according to any of the methods for preparing compositions disclosed herein.

Where elements are presented as lists, e.g., in Markush group format, it is to be understood that each subgroup of the elements is also disclosed, and any element(s) can be removed from the group. It is also noted that the term “comprising” is intended to be open and permits the inclusion of additional elements or steps. It should be understood that, in general, where the invention, or aspects of the invention, is/are referred to as comprising particular elements, features, steps, etc., certain embodiments of the invention or aspects of the invention consist, or consist essentially of, such elements, features, steps, etc. For purposes of simplicity those embodiments have not been specifically set forth in haec verba herein. Thus for each embodiment of the invention that comprises one or more elements, features, steps, etc., the invention also provides embodiments that consist or consist essentially of those elements, features, steps, etc.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or more of the claims. Any embodiment, element, feature, application, or aspect of the compositions and/or methods of the invention can be excluded from any one or more claims. For purposes of brevity, all of the embodiments in which one or more elements, features, purposes, or aspects is excluded are not set forth explicitly herein.

### REFERENCES

The following references are herein incorporated by reference:
4,461,281
5,131,382
5,454,827
5,531,744
5,749,893
5,785,647
5,851,212
5,897,523
5,899,914
5,938,678
6,010,054
6,048,339
6,053,907
6,077,287
6,183,433
6,645,218
6,656,195
RE39,152

1. An apparatus for removing tissue from a body cavity, the apparatus comprising:
an inner sleeve;
a middle sleeve comprising an aperture that exposes a portion of the inner sleeve;
an outer sleeve; and
an irrigation conduit, wherein:
the irrigation conduit comprises a first end proximal to the aperture of the middle sleeve; and
the inner sleeve is configured to move relative to the middle sleeve.

2. The apparatus of claim 1 wherein the irrigation conduit is disposed between the middle sleeve and the outer sleeve.

3. The apparatus of claim 1 wherein the irrigation conduit is integral to the outer sleeve.

4. The apparatus of claim 1 wherein the outer sleeve comprises a flexible portion proximal to the aperture of the middle sleeve.

5. The apparatus of claim 4 wherein the outer sleeve comprises a continuous wall in the flexible portion.

6. The apparatus of claim 1 wherein the outer sleeve comprises polyethylene, polypropylene, or PTFE.

7. The apparatus of claim 1 wherein the inner sleeve rotates within the middle sleeve.

8. The apparatus of claim 1 wherein the inner sleeve moves axially within the middle sleeve.

9. The apparatus of claim 1 wherein the inner sleeve comprises a blade exposed by the aperture.

10. The apparatus of claim 1 wherein the outer sleeve comprises a control member configured to articulate the outer sleeve, the middle sleeve and the inner sleeve.

11. The apparatus of claim 10 wherein the control member is a cable, wire or ribbon.

12. The apparatus of claim 1 wherein the aperture comprises a sharp edge.

13. A method of removing tissue from a body cavity, the method comprising:
providing an apparatus comprising:
an inner sleeve;
a middle sleeve comprising an aperture that exposes a portion of the inner sleeve;
an outer sleeve; and
an irrigation conduit, wherein:
the irrigation conduit comprises a first end proximal to the aperture of the middle sleeve; and
the inner sleeve is configured to move relative to the middle sleeve;
inserting the apparatus into the body cavity;
excising tissue by engaging tissue between the inner sleeve and the middle sleeve;
irrigating the body cavity with fluid from the irrigation conduit; and
removing the excised tissue.

14. The method of claim 13 further comprising providing suction to the inner sleeve.

15. The method of claim 13 wherein the body cavity is an ear, nose or throat cavity.

16. The method of claim 13 further comprising rotating the inner sleeve relative to the middle sleeve.

17. The method of claim 13 further comprising moving the inner sleeve axially relative to the middle sleeve.

18. The method of claim 13 further comprising articulating the outer sleeve, the middle sleeve and the inner sleeve.

19. The method of claim 13 wherein the outer sleeve comprises a control member, and wherein the outer sleeve, the middle sleeve and the inner sleeve are articulated via the control member.

20. The method of claim 19 wherein the control member comprises a cable, wire or ribbon.
